(19) United States
(12) Patent Application Publication
(10) Pub. No.: US 2018/0008524 A1
(43) Pub. Date: Jan. 11, 2018

(54) BLEACHING AGENT AND METHOD FOR GENTLE OXIDATIVE LIGHTENING OF KERATIN FIBRES

(71) Applicant: Henkel AG & Co. KGaA, Duesseldorf (DE)

(72) Inventors: Bernd Anderheggen, Moenchengladbach (DE); Burkhard Mueller, Duesseldorf (DE)

(73) Assignee: Henkel AG & Co. KGaA, Duesseldorf (DE)

(21) Appl. No.: 15/643,475

(22) Filed: Jul. 7, 2017

(30) Foreign Application Priority Data
Jul. 7, 2016 (DE) 10 2016 212 430.7

(51) Int. Cl. A61K 8/73; A61K 8/362; A61K 8/23; A61Q 5/08; A61K 8/44

(52) U.S. Cl. CPC A61K 8/731; A61Q 5/08; A61K 8/44; A61K 8/23; A61K 8/362; A61K 2800/882

(57) ABSTRACT

The subject of the present invention is the use of a combination of cellulose 2-[2-hydroxy-3-(trimethylammonio)propoxy]ethyl ether chloride and at least one amino acid, selected from arginine, lysine, histidine or at least one of the salts of these amino acids, in a bleaching powder in order to reduce the damage to keratin fibres caused by an oxidative bleaching treatment.

### CROSS-REFERENCE TO RELATED APPLICATION

This application claims priority to German Patent Application No. 10 2016 212 430.7, filed Jul. 7, 2016, which is incorporated herein by reference in its entirety.

### TECHNICAL FIELD

The present disclosure relates to bleaching powders which serve as agents for lightening keratin fibres, in particular human hair. The present disclosure also relates to the use of the agents for the gentle bleaching or oxidative lightening of human hair, and to a multi-component packaging unit (kit-of-parts) for lightening keratin fibres, which comprises a bleaching powder as contemplated herein and, separately therefrom, an oxidising agent preparation.

### BACKGROUND

A method for the oxidative lightening of keratin fibres with use of the aforementioned bleaching powder and the kit comprising this bleaching powder is also described.

The bleaching powder is an anhydrous, powder-like oxidising agent preparation containing at least one persalt or percarbonate.

Many consumers have always desired to lighten their own hair because a blond hair colour is considered to be attractive and desirable from a fashion point of view. Various bleaching agents with different bleaching power are commercially available for this purpose. The oxidising agents present in these products are capable of lightening hair fibres by oxidatively degrading the hair's own colorant, melanin. For a moderate bleaching effect, the use of hydrogen peroxide—optionally with use of ammonia or other alkalising agents—as sole oxidising agent is sufficient. In order to attain a stronger bleaching effect, a mixture of hydrogen peroxide and at least one compound, selected from percarbonates and persalts, in particular peroxydisulfate salts and/or peroxymonosulfate salts, is usually used. To intensify the bleaching effect, the agents contain higher use concentrations of hydrogen peroxide and percarbonates or persalts, in particular persulfates. Dark, dark brown, or black hair can thus be lightened by 4 to 6 shades in a single step. The hydrogen peroxide and the percarbonates or persalts are stored separately from one another until they are used so as not to prematurely deactivate the percarbonates or persalts. The hydrogen peroxide component, which comprises an aqueous solution of hydrogen peroxide, has an acidic pH value, in particular a pH value from about 2.5 to about 5.5, in particular from about 3 to about 5, measured in each case at 20° C., for stabilisation of the hydrogen peroxide.

For the melanin-degrading effect of the hydrogen peroxide and the bleaching effect on the keratin fibres, it is advantageous however if the mixture to be used formed of hydrogen peroxide solution and persalt has an alkaline pH value, which preferably lies in the range from about 8 to about 12, particularly preferably in the range from about 8.5 to about 11.5, extremely preferably in the range from about 9 to about 10.5, measured in each case at 20° C.

There are a number of possibilities for setting an alkaline pH value of the lightening mixture to be used:

the bleaching powder, besides the at least one persalt or percarbonate, contains at least one powder-like alkalising agent in such a total amount that the mixture to be used has the desired alkaline pH value; or
the hydrogen peroxide solution is not only combined with the bleaching powder, but additionally with an alkalising agent preparation to form the mixture for use.

If the alkalising agent preparation and/or the bleaching powder is/are mixed with oxidation dye precursor products and/or substantive dyes, the hair can be coloured at the same time. Corresponding 3-component hair dyes are offered in particular for consumers having very dark melanin-rich hair.

However, the lightening is also accompanied by damage to the hair, since not only the pigments of the hair, but also the structural components of the hair are oxidatively damaged. Depending on the extent of the damage, it ranges from coarse, brittle and tangled hair, over a reduced resistance and tear resistance of the hair, to hair breakage. The greater the amount of the used hydrogen peroxide and optionally persalts or percarbonates, the greater generally is the damage therefore caused to the keratin fibres.

In order to minimise the damage to the hair and at least partially compensate for the damaging effect of the oxidising agents, it is increasingly attempted to formulate hair-lightening and hair-colouring agents containing persalt(s) with a higher content of oils.

In the prior art, for example, bleaching agent suspensions are described which constitute anhydrous suspensions of fine-particle persalts or percarbonates, solid at 25° C. and 1013 mbar, in an oil or an oil mixture, which can be thickened optionally with an oil-gelling agent; see EP 0778020, EP 1034777 and EP 1380287. A disadvantage here is that it is difficult to prepare a homogeneous mixture of this very hydrophobic paste and of the hydrogen peroxide preparation (which has a very high water content) and, where applicable, the alkalising agent preparation, which usually also contains water, said mixture requiring vigorous shaking or stirring for a longer period of time. The preparation of a bleaching agent suspension is also technically more complex than the preparation of a powder-like persalt mixture.

### BRIEF SUMMARY

Bleaching powders, methods for lightening keratin fibers, and multi-component packaging units are provided herein. In an embodiment, a bleaching powder includes: a) at least one oxidising agent selected from sodium percarbonates, inorganic salts of a peroxysulfuric acid, or mixtures hereof, b) cellulose 2-[2-hydroxy-3-(trimethylammonio)propoxy]ethyl ether chloride, c) at least one amino acid selected from arginine, lysine, histidine, or at least one of the salts of these amino acids, and d) at least one complexing agent selected from the following acids and/or alkali metal salts thereof: ethylenediaminetetraacetic acid (EDTA); N-hydroxyethylethylenediaminetriacetic acid; aminotrimethylenephosphonic acid; diethylenetriaminepentaacetic acid; lauroyl ethylene diaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder.

In another embodiment, a method for lightening keratin fibres includes mixing a bleaching powder with an oxidation composition. The bleaching powder includes a) at least one oxidising agent selected from sodium percarbonates, inorganic salts of a peroxysulfuric acid, or mixtures hereof, b) cellulose 2-[2-hydroxy-3-(trimethylammonio)propoxy]ethyl ether chloride, c) at least one amino acid selected from arginine, lysine, histidine, or at least one of the salts of these amino acids, and d) at least one complexing agent selected from the following acids and/or alkali metal salts thereof: ethylenediaminetetraacetic acid (EDTA); N-hydroxy ethyl ethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder. The oxidation composition includes, in each case in relation to its weight, from about 50-about 96% by weight of hydrogen peroxide, at least one pH adjustor in such an amount that the oxidation composition has a pH value in the range of from about 2.5 to about 5.5, measured at 20° C. The mixture of the bleaching powder and the oxidation composition is applied to the keratin-comprising fibres directly after mixing, wherein the mixture is left on the fibres for from about 5 to about 60 minutes. The fibres are rinsed with water with the mixture optionally washed out using a surfactant-containing cleansing agent.

In another embodiment, a multi-component packaging unit (kit-of-parts) for lightening keratin fibres includes at least two components that are packaged separately from one another. The first component (I) is a bleaching powder including a) at least one oxidising agent selected from sodium percarbonates, inorganic salts of a peroxysulfuric acid, or mixtures hereof, b) cellulose 2-[2-hydroxy-3-(trimethylammonio)propoxy]ethyl ether chloride, c) at least one amino acid selected from arginine, lysine, histidine, or at least one of the salts of these amino acids, and d) at least one complexing agent selected from the following acids and/or alkali metal salts thereof: ethylenediaminetetraacetic acid (EDTA); N-hydroxyethyl ethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder. The second component (II) is an oxidation composition which includes, in each case in relation to its weight, from about 50-about 96% by weight of water and from about 0.5-about 20% by weight of hydrogen peroxide and has a pH value in the range of from about 2.5 to about 5.5, measured at 20° C.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide agents for lightening or bleaching keratin fibres, in particular human hair, which damage the keratin fibres to a minimal extent and which can be easily produced and handled. The reduction of fibre damage should be attained here to the greatest possible extent not by oils, but by alternative nourishing active substances. These objects were achieved surprisingly by the subject matter of the claims.

The term keratin-containing fibres or keratin fibres is understood as contemplated herein to mean furs, wool, feathers and in particular human hair. Although the agents as contemplated herein are primarily suitable for bleaching and/or lightening keratin-containing fibres, there is in principle nothing against a use also in other fields.

A suitable parameter for the quantification of the fibre damage, in particular hair damage, is the measurement of tensile strength (Young's modulus) of the keratin fibres.

A first subject matter of the present disclosure is a bleaching powder, containing

a. at least one oxidising agent selected from sodium percarbonates and inorganic salts of a peroxysulfuric acid, and mixtures hereof,
b. also cellulose 2-[2-hydroxy-3-(trimethylammonio)propoxy]ethyl ether chloride,
c. also at least one amino acid, selected from arginine, lysine, histidine or at least one of the salts of these amino acids,
d. at least one complexing agent, selected from the following acids and/or alkali metal salts thereof: ethylenediaminetetraacetic acid (EDTA); N-hydroxyethyl ethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, and mixtures hereof, in a total amount of from about 0.1-about 1.4% by weight, preferably from about 0.2-about 1.4% by weight, particularly preferably from about 0.5-about 1.4% by weight, in each case in relation to the weight of the bleaching powder.

The terms “powder” or “powder-like” are to be understood, as contemplated herein, to mean an administration form formed of individual particles which, at 20° C. and 1013 mbar, is solid and can be poured, the individual particles having particle sizes in the range of from about 0.1 μm to at most about 1.6 mm. The particle sizes can be determined preferably by employing laser diffraction measurement in accordance with ISO 13320-1 (2009). As appropriate, the grain size of the particles can be adapted to the requirements of the bleaching powder by physical treatment, such as sieving, pressing, granulation or palletisation, or by the addition of specific auxiliaries, so as to enable for example an improved miscibility of the individual powder constituents or the miscibility of the bleaching powder with a hydrogen peroxide preparation.

Bleaching powders that are preferred as contemplated herein have a bulk density in the range of from about 500 to about 1000 g/l (grams/litre), preferably from about 550 to about 900 g/l, particularly preferably from about 600 to about 820 g/l. The bulk density is determined preferably in accordance with EN ISO 60 (version 01/2000) or DIN ISO 697 (version 01/1984).

Unless specified otherwise, all specified temperatures relate to a pressure of 1013 mbar.

The bleaching powder as contemplated herein contains, as first essential constituent, at least one oxidising agent which is selected from sodium percarbonates and inorganic salts of a peroxysulfuric acid and mixtures thereof.

The term sodium percarbonates is understood to mean sodium carbonate-hydrogen peroxide complexes. Commercially conventional sodium percarbonate has the average composition 2 Na₂CO₃.3H₂O₂. Sodium percarbonate is present in the form of a white, water-soluble powder, which easily decays into sodium carbonate and “active” oxygen having a bleaching and oxidising effect.

Peroxysulfuric acids are understood to mean peroxydisulfuric acid and peroxymonosulfuric acid (Caro's acid).

The at least one inorganic salt of a peroxysulfuric acid is preferably selected from ammonium peroxydisulfate, alkali metal peroxydisulfates, ammonium peroxymonosulfate, alkali metal peroxymonosulfates and alkali metal hydrogen peroxymonosulfates. Ammonium peroxydisulfate, potassium peroxydisulfate, sodium peroxydisulfate and potassium hydrogen peroxymonosulfate are particularly preferred. Within the scope of the works for the present disclosure, it has also proven to be particularly preferable if the bleaching powder as contemplated herein contains at least two different peroxydisulfates. Preferred peroxydisulfates are, here, combinations of ammonium peroxydisulfate and potassium peroxydisulfate and/or sodium peroxydisulfate.

Preferred bleaching powders as contemplated herein contain at least one oxidising agent, which is selected from sodium percarbonates and inorganic salts of a peroxysulfuric acid and mixtures hereof, in a total amount of from about 5-about 85% by weight, preferably from about 10-about 75% by weight, particularly preferably from about 15-about 65% by weight, and extremely preferably from about 20-about 55% by weight, in each case in relation to the weight of the bleaching powder.

The bleaching powder as contemplated herein contains, as second essential constituent, cellulose 2-[2-hydroxy-3-(trimethylammonio)propoxy]ethyl ether chloride. The cellulose 2-[2-hydroxy-3-(trimethylammonio)propoxy]ethyl ether chloride is preferably contained in a total amount of from about 0.01-about 1.00% by weight, particularly preferably from about 0.10-about 0.60% by weight, more particularly preferably from about 0.20-about 0.48, and extremely preferably from about 0.30-about 0.47% by weight, in each case in relation to the weight of the bleaching powder.

The bleaching powder as contemplated herein also contains, as third essential constituent, at least one amino acid, selected from arginine, lysine, histidine or at least one of the salts of these amino acids. Arginine is extremely preferred. Mixtures of arginine and lysine can be particularly preferred as contemplated herein. The salts of arginine, lysine or histidine that are preferably suitable as contemplated herein are the ammonium salts, alkali metal salts, and alkaline earth metal salts, in particular the lithium, sodium, potassium, magnesium and calcium salts, and in addition the hydrohalides, in particular the hydrochlorides, and mixtures of these salts. An amino acid salt that is particularly preferred as contemplated herein is lysine hydrochloride. The amino acids suitable as contemplated herein, selected from arginine, lysine, histidine and salts thereof, can also contain water of crystallisation.

Bleaching powders that are preferred as contemplated herein contain at least one amino acid, selected from arginine, lysine, histidine or at least one salt of these amino acids, in a total amount, converted to the mass of free amino acid, of from about 0.1-about 7% by weight, preferably from about 0.2-about 5% by weight, particularly preferably from about 0.5-about 2.5% by weight, extremely preferably from about 1-about 2% by weight, in each case in relation to the weight of the bleaching powder.

The combination of cellulose 2-[2-hydroxy-3-(trimethylammonio)propoxy]ethyl ether chloride and arginine has proven to be extremely preferable as contemplated herein. It has surprisingly been found that a significant reduction of the hair damage caused by the oxidative hair lightening is provided by the combination of cellulose 2-[2-hydroxy-3-(trimethylammonio)propoxy]ethyl ether chloride and arginine.

Bleaching powders that are particularly preferred as contemplated herein contain cellulose 2-[2-hydroxy-3-(trimethylammonio)propoxy]ethyl ether chloride in a total amount of from about 0.01-about 1.00% by weight, particularly preferably from about 0.10-about 0.60% by weight, more particularly preferably from about 0.20-about 0.48% by weight, extremely preferably from about 0.30-about 0.47% by weight, and also at least one amino acid selected from arginine, lysine, or at least one salt of these amino acids, in a total amount, converted to the mass of free amino acid, of from about 0.1-about 7% by weight, preferably from about 0.2-about 5% by weight, particularly preferably from about 0.5-about 2.5% by weight, extremely preferably from about 1-about 2% by weight, wherein all specified values relate to the weight of the bleaching powder.

Bleaching powders that are preferred as contemplated herein additionally contain at least one inorganic alkalising agent which is solid at 20° C. and 1013 mbar, including at least one sodium silicate or sodium metasilicate having a molar SiO₂/Na₂O ratio of ≧about 2, preferably from about 2.5-about 3.5, in a total amount of from about 0.1 to about 50% by weight, preferably from about 5 to about 40% by weight, in each case in relation to the weight of the bleaching powder.

Bleaching powders that are preferred as contemplated herein contain at least one inorganic alkalising agent which is solid at 20° C. and 1013 mbar, preferably in a total amount of from about 1-about 60% by weight, preferably from about 5-about 55% by weight, particularly preferably from about 10-about 50% by weight, extremely preferably from about 15-about 45% by weight, in each case in relation to the weight of the bleaching powder.

Besides the at least one sodium silicate or sodium metasilicate having a molar SiO₂/Na₂O ratio of ≧about 2, preferably from about 2.5-about 3.5, in a total amount of from about 0.1 to about 50% by weight, preferably from about 5 to about 40% by weight, in each case in relation to the weight of the bleaching powder, further inorganic alkalising agents which are solid at 20° C. and 1013 mbar and which are particularly preferred as contemplated herein are selected as optional alkalising agent from alkaline earth metal silicates, alkaline earth metal hydroxide carbonates, alkaline earth metal carbonates, alkaline earth metasilicates, alkali metal hydroxides, alkaline earth metal hydroxides, (earth) alkali metal phosphates and (earth) alkali metal hydrogen phosphates, and mixtures of these substances. Besides the at least one obligatory sodium silicate or sodium metasilicate, in each case with a molar SiO₂/Na₂O ratio of ≧about 2, preferably from about 2.5-about 3.5, inorganic alkalising agents which are solid at 20° C. and 1013 mbar and which are particularly preferred as contemplated herein are selected from magnesium hydroxide carbonates and mixtures of these alkalising agents. Magnesium hydroxide carbonates which are preferred as contemplated herein are those with the formula MgCO₃.Mg(OH)₂.2H₂O and those with the formula MgCO₃.Mg(OH)₂. Magnesium hydroxide carbonate with the formula MgCO₃.Mg(OH)₂. is particularly preferred as contemplated herein.

Bleaching powders that are particularly preferred as contemplated herein contain, in each case in relation to their total weight, from about 0.1 to about 50% by weight, preferably from about 5 to about 40% by weight, of sodium silicates having a molar SiO₂/Na₂O ratio of ≧about 2, preferably from about 2.5 to about 3.5, and from about 2-about 20% by weight, preferably from about 5-about 15% by weight, particularly preferably from about 8-about 25% by weight, of magnesium hydroxide carbonate as inorganic alkalising agent solid at 20° C. and 1013 mbar.

Bleaching powders that are extremely preferred as contemplated herein contain, in each case in relation to their total weight, from about 0.1 to about 50% by weight, preferably from about 5 to about 40% by weight, of sodium silicates having a molar SiO₂/Na₂O ratio of ≧about 2, preferably from about 2.5 to about 3.5, and from about 2-about 20% by weight, preferably from about 5-about 15% by weight, particularly preferably from about 10-about 13% by weight, of magnesium hydroxide carbonate with the formula MgCO₃.Mg(OH)₂ as inorganic alkalising agent solid at 20° C. and 1013 mbar.

Provided the bleaching powder as contemplated herein or the bleaching powder that is preferred as contemplated herein contains one or more inorganic carbonates, whether as alkalising agent or as oxidising agent in the form of sodium carbonate-hydrogen peroxide complexes, the content thereof is preferably selected such that the molar CO₃² total concentration in the mixture for use having the above-discussed oxidation composition (Ox) is at least 0.015 mol/100 grams of mixture for use.

Provided the bleaching powder as contemplated herein or the bleaching powder that is preferred as contemplated herein contains one or more inorganic carbonates, whether as alkalising agent or as oxidising agent in the form of sodium carbonate-hydrogen peroxide complexes, the content thereof is particularly preferably selected such that the molar CO₃² total concentration in the mixture for use having the above-discussed oxidation composition (Ox) is mathematically at least four times greater than the total concentration of proton donors.

Provided the bleaching powder as contemplated herein or the bleaching powder that is preferred as contemplated herein contains one or more inorganic carbonates, whether as alkalising agent or as oxidising agent in the form of sodium carbonate-hydrogen peroxide complexes, the content thereof is extremely preferably selected such that the molar CO₃² total concentration in the ready-to-use mixture having the above-discussed oxidation composition (Ox) is at least 0.015 mol/100 grams of mixture for use and is mathematically at least four times greater than the total concentration of proton donors.

The bleaching powders as contemplated herein preferably have a water content of from about 0 to about 8% by weight, preferably from about 0.1 to about 5% by weight, particularly preferably from about 0.5 to about 3% by weight of water, in each case in relation to the weight of the bleaching powder. These values relate to the content of free water. What is not considered is the content of molecularly bound water or water of crystallisation, which individual powder constituents may have. The water content can be determined by employing Karl-Fischer titration, for example on the basis of ISO 4317 (version 2011-12).

In a preferred embodiment the bleaching powder as contemplated herein also contains at least one dicarboxylic acid having 2 to 10 carbon atoms, particularly preferably selected from succinic acid, malic acid, oxalic acid, malonic acid, adipic acid, pimelic acid, suberic acid, azelaic acid, sebacic acid, maleic acid, fumaric acid, D-tartaric acid, L-tartaric acid, meso-tartaric acid, racemic acid, alpha-ketoglutaric acid, beta-ketoglutaric acid, oxaloacetic acid and/or at least one salt of these acids and mixtures of these compounds, wherein the at least one dicarboxylic acid having 2 to 10 carbon atoms is extremely preferably selected from succinic acid, malic acid and maleic acid and salts thereof.

Salts of dicarboxylic acids having 2 to 10 carbon atoms that are preferred as contemplated herein are selected from the mono salts and di salts of the anions of succinic acid, malic acid, oxalic acid, malonic acid, adipic acid, pimelic acid, suberic acid, azelaic acid, sebacic acid, maleic acid, fumaric acid, D-tartaric acid, L-tartaric acid, meso-tartaric acid, racemic acid, alpha-ketoglutaric acid, beta-ketoglutaric acid, oxaloacetic acid with ammonium ions, alkali metal ions, alkaline earth metal ions and the ions of basic amino acids, such as arginine, lysine and histidine, in particular with lithium, sodium, potassium, magnesium and calcium ions, and mixtures of these salts.

Succinic acid, which is particularly preferred as contemplated herein, has, at 1013 mbar, a melting point in the range of 185-187° C., i.e. is a solid at 20° C. Salts of succinic acid which are suitable as contemplated herein are selected from the succinates and hydrogen succinates of ammonium ions, alkali metal ions, alkaline earth metal ions and the ions of basic amino acids, such as arginine, lysine and histidine, in particular the lithium, sodium, potassium, magnesium and calcium ions, or the succinates and hydrogen succinates of basic amino acids, such as arginine, lysine and/or histidine, for example arginine succinate, and mixtures of these salts. The specified salts of succinic acid can also contain bound water of crystallisation, in particular the sodium succinate hexahydrate, which is particularly preferred as contemplated herein.

Malic acid, which is particularly preferred as contemplated herein, is optically active. Racemic DL-malic acid has, at 1013 mbar, a melting point in the range of 131-132° C., i.e. is a solid at 20° C. The enantiomers D-malic acid and L-malic acid each have, at 1013 mbar, a melting point in the range of 100-101° C. For cost reasons, racemic DL-malic acid is preferred.

Salts of malic acid suitable as contemplated herein are selected from the malates and hydrogen malates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts, in particular disodium malate and dipotassium malate, but also calcium malate. The specified salts of malic acid suitable as contemplated herein can contain bound water of crystallisation, in particular the disodium malate hemihydrate and the disodium malate trihydrate.

Oxalic acid, which is preferred as contemplated herein, has, at 1013 mbar, a melting point of 189.5° C. (anhydrous) or as dihydrate a melting point of 101.5° C. Salts of oxalic acid suitable as contemplated herein are selected from the oxalates and hydrogen oxalates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Malonic acid, which is preferred as contemplated herein, has, at 1013 mbar, a melting point of 135° C. Salts of malonic acid suitable as contemplated herein are selected from the malates and hydrogen malates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Adipic acid, which is preferred as contemplated herein, has, at 1013 mbar, a melting point of 152° C. Salts of adipic acid suitable as contemplated herein are selected from the adipates and hydrogen adipates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Pimelic acid, which is preferred as contemplated herein, has, at 1013 mbar, a melting point of 105° C. Salts of pimelic acid suitable as contemplated herein are selected from the pimelates and hydrogen pimelates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Suberic acid, which is preferred as contemplated herein, has, at 1013 mbar, a melting point of 144° C. Salts of suberic acid suitable as contemplated herein are selected from the suberates and hydrogen suberates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Azelaic acid, which is preferred as contemplated herein, has, at 1013 mbar, a melting point of 106° C. Salts of azelaic acid suitable as contemplated herein are selected from the azelates and hydrogen azelates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Sebacic acid, which is preferred as contemplated herein, has, at 1013 mbar, a melting point of 134.5° C. Salts of sebacic acid suitable as contemplated herein are selected from the sebacates and hydrogen sebacates of ammonium ions, alkali metal ions, alkaline earth metal ions, and the ions of basic amino acids, such as arginine, lysine and histidine, in particular of lithium, sodium, potassium, magnesium and calcium ions, and mixtures of these salts.

Maleic acid, which is particularly preferred as contemplated herein, has, at 1013 mbar, a melting point of from about 130 to about 131° C. (from ethanol or benzene) and of from about 138 to about 139° C. (from water). Salts of maleic acid suitable as contemplated herein are selected from the maleates and hydrogen maleates of ammonium ions, alkali metal ions, and alkaline earth metal ions, in particular of lithium, sodium, potassium, magnesium and calcium ions, and mixtures of these salts.

Fumaric acid, which is particularly preferred as contemplated herein, has, at 1013 mbar, a melting point of 287° C. in a heat-sealed tube; fumaric acid sublimes at 200° C. Salts of fumaric acid suitable as contemplated herein are selected from the fumarates and hydrogen fumarates of ammonium ions, alkali metal ions, and alkaline earth metal ions, in particular of lithium, sodium, potassium, magnesium and calcium ions, and mixtures of these salts.

D-tartaric acid (laevorotatory), which is particularly preferred as contemplated herein, has, at 1013 mbar, a melting point of 168-170° C. Salts of D-tartaric acid suitable as contemplated herein are selected from the tartrates and hydrogen tartrates of ammonium ions, alkali metal ions, and alkaline earth metal ions, in particular of lithium, sodium, potassium, magnesium and calcium ions, and mixtures of these salts.

L-tartaric acid (dextrorotatory), which is particularly preferred as contemplated herein, has, at 1013 mbar, a melting point of 168-170° C. Salts of L-tartaric acid suitable as contemplated herein are selected from the tartrates and hydrogen tartrates of ammonium ions, alkali metal ions, and alkaline earth metal ions, in particular of lithium, sodium, potassium, magnesium and calcium ions, and mixtures of these salts.

Meso-tartaric acid, which is particularly preferred as contemplated herein, has, at 1013 mbar, a melting point of 140° C. Salts of meso-tartaric acid suitable as contemplated herein are selected from the tartrates and hydrogen tartrates of ammonium ions, alkali metal ions, and alkaline earth metal ions, in particular of lithium, sodium, potassium, magnesium and calcium ions, and mixtures of these salts.

Racemic acid, which is particularly preferred as contemplated herein, is the racemic mixture of D-tartaric acid and L-tartaric acid. Racemic acid has, at 1013 mbar, a melting point of 206° C. Salts of racemic acid suitable as contemplated herein are selected from the tartrates and hydrogen tartrates of ammonium ions, alkali metal ions, and alkaline earth metal ions, in particular of lithium, sodium, potassium, magnesium and calcium ions, and mixtures of these salts.

Alpha-ketoglutaric acid, which is particularly preferred as contemplated herein, has, at 1013 mbar, a melting point of 112-116° C. Salts of alpha-ketoglutaric acid suitable as contemplated herein are selected from the alpha-ketoglutarates and alpha-keto hydrogen glutarates of ammonium ions, alkali metal ions, and alkaline earth metal ions, in particular of lithium, sodium, potassium, magnesium and calcium ions, and mixtures of these salts.

Beta-ketoglutaric acid, which is particularly preferred as contemplated herein, has, at 1013 mbar, a melting point of 122° C.; it melts with decomposition. Salts of beta-ketoglutaric acid suitable as contemplated herein are selected from the beta-ketoglutarates and beta-keto hydrogen glutarates of ammonium ions, alkali metal ions, and alkaline earth metal ions, in particular of lithium, sodium, potassium, magnesium and calcium ions, and mixtures of these salts.

Oxaloacetic acid, which is particularly preferred as contemplated herein, has, at 1013 mbar, a melting point of 161° C. Salts of oxaloacetic acid suitable as contemplated herein are selected from the oxaloacetates and oxalo hydrogen acetates of ammonium ions, alkali metal ions, and alkaline earth metal ions, in particular of lithium, sodium, potassium, magnesium and calcium ions, and mixtures of these salts.

Bleaching powders which are preferred as contemplated herein contain the at least one dicarboxylic acid having 2 to 10 carbon atoms, selected from succinic acid, malic acid, oxalic acid, malonic acid, adipic acid, pimelic acid, suberic acid, azelaic acid, sebacic acid, and/or at least one salt of these acids, in a total amount, converted to the mass of free dicarboxylic acid, of from about 0.03-about 7% by weight, preferably from about 0.1-about 5% by weight, particularly preferably from about 0.5-about 3% by weight, extremely preferably from about 0.9-about 1.5% by weight, in each case in relation to the weight of the bleaching powder.

Further bleaching powders that are preferred as contemplated herein contain succinic acid and/or at least one salt of succinic acid in a total amount, converted to the mass of free dicarboxylic acid, of from about 0.03-about 7% by weight, preferably from about 0.1-about 5% by weight, particularly preferably from about 0.5-about 3% by weight, extremely preferably from about 0.9-about 1.5% by weight, in each case in relation to the weight of the bleaching powder.

Further bleaching powders that are preferred as contemplated herein contain malic acid and/or at least one salt of malic acid in a total amount, converted to the mass of free dicarboxylic acid, of from about 0.03-about 7% by weight, preferably from about 0.1-about 5% by weight, particularly preferably from about 0.5-about 3% by weight, extremely preferably from about 0.9-about 1.5% by weight, in each case in relation to the weight of the bleaching powder.

In order to dedust the bleaching powders as contemplated herein, at least one dedusting agent can be added, which in particular is selected from at least one oil, in particular selected from paraffin oil, silicone oil or ester oil, and mixtures of these oils.

Bleaching powders that are preferred as contemplated herein additionally contain at least one oil in a total amount of from about 0.1-about 15% by weight, preferably from about 0.5-about 10% by weight, particularly preferably from about 1-about 8% by weight, extremely preferably from about 2-about 6% by weight, in each case in relation to the weight of the bleaching powder.

Oils that are preferred as contemplated herein are selected from natural and synthetic hydrocarbons, particularly preferably from paraffin oils, C₁₈-C₃₀ isoparaffins, in particular isoeicosane, polyisobutenes, and polydecenes, further selected from C₈-C₁₆ isoparaffins, in particular from isodecane, isododecane, isotetradecane, and isohexadecane and mixtures thereof, and also 1,3-di-(2-ethyl hexyl)-cyclohexane.

Further oils that are preferred as contemplated herein are selected from the benzoic acid esters of linear or branched C8-C22 alkanols. Benzoic acid C12-C15 alkyl esters are particularly preferred.

Further oils that are preferred as contemplated herein are selected from fatty alcohols having 6-30 carbon atoms, which are unsaturated or branched and saturated or branched and unsaturated. Preferred alcohol oils are 2-hexyldecanol, 2-octyldodecanol, 2-ethylhexyl alcohol, and isostearyl alcohol.

Further cosmetic oils that are preferred as contemplated herein are selected from the triglycerides (=triple esters of glycerol) of linear or branched, saturated or unsaturated, optionally hydroxylated C8-30 fatty acids. The use of natural oils, for example amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, linseed oil, macadamia nut oil, corn oil, almond oil, manila oil, evening primrose oil, olive oil, palm oil, palm kernel oil, para nut oil, pecan nut oil, peach kernel oil, rapeseed oil, castor oil, sea buckthorn pulp oil, sea buckthorn seed oil, sesame oil, soy oil, sunflower oil, grapeseed oil, walnut oil, wild rose oil, wheat germ oil, and the liquid components of coconut oil and the like, can be particularly preferred. Synthetic triglyceride oils are also preferred, however, in particular capric/caprylic triglycerides.

Further cosmetic oils that are particularly preferred as contemplated herein are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl)succinate.

Further cosmetic oils that are particularly preferred as contemplated herein are selected from esters of linear or branched, saturated or unsaturated fatty alcohols having 2-30 carbon atoms with linear or branched, saturated or unsaturated fatty acids having 2-30 carbon atoms, which can be hydroxylated. These preferably include 2-hexyldecyl stearate, 2-hexyldecyl laurate, isodecyl neopentanoate, isononyl isononanoate, 2-ethylhexyl palmitate, and 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid 2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Further cosmetic oils that are preferred as contemplated herein are selected from the addition products of 1 to 5 propylene oxide units with mono- or polyvalent C₈₋₂₂ alkanols such as octanol, decanol, decanediol, lauryl alcohol, myristyl alcohol, and stearyl alcohol, e.g. PPG-2 Myristyl Ether and PPG-3 Myristyl Ether. Further cosmetic oils that are preferred as contemplated herein are selected from addition products of at least 6 ethylene oxide and/or propylene oxide units with mono- or polyvalent C₃₋₂₂ alkanols such as glycerol, butanol, butanediol, myristyl alcohol, and stearyl alcohol, which can be esterified if desired, e.g. PPG-14 Butyl Ether, PPG-9 Butyl Ether, PPG-10 Butanediol, PPG-15 Stearyl Ether, and Glycereth-7 diisonoanoate.

Further cosmetic oils that are preferred as contemplated herein are selected from C₈-C₂₂ fatty alcohol esters of monovalent or polyvalent C₂-C₇ hydroxycarboxylic acids, in particular the esters of glycolic acid, lactic acid, malic acid, tartaric acid, citric acid, and salicylic acid, for example C₁₂-C₁₅ alkyl lactate.

Further cosmetic oils that are preferred as contemplated herein are selected from symmetrical, asymmetrical, or cyclic esters of carbonic acid with C₃₋₂₂ alkanols, C₃₋₂₂ alkanediols, or C₃₋₂₂ alkanetriols, e.g. dicaprylyl carbonate, or the esters according to DE 19756454 A1, in particular glycerol carbonate.

Further cosmetic oils that are suitable in accordance with the present disclosure are selected from the silicone oils that include, for example, decamethylcyclopentasiloxane, dodecamethylcyclohexasiloxane, dimethylpolysiloxane and methylphenylpolysiloxane, but also hexamethyldisiloxane, octamethyltrisiloxane and decamethyltetrasiloxane.

Mixtures of the aforementioned oils can be used extremely preferably as contemplated herein.

Preferred bleaching powders as contemplated herein are exemplified in that the cosmetic oil is selected from natural and synthetic hydrocarbons, particularly preferably from paraffin oils, C₁₈-C₃₀ isoparaffins, in particular isoeicosane, polyisobutenes, and polydecenes, C₈-C₁₆ isoparaffins, and 1,3-di-(2-ethylhexyl)cyclohexane; benzoic acid esters of linear or branched C₈₋₂₂ alkanols; fatty alcohols having 6-30 carbon atoms, which are unsaturated or branched and saturated or branched and unsaturated; triglycerides of linear or branched, saturated or unsaturated, optionally hydroxylated C₈₋₃₀ fatty acids, in particular natural oils; dicarboxylic acid esters of linear or branched C₂-C₁₀ alkanols; esters of linear or branched, saturated or unsaturated fatty alcohols having 2-30 carbon atoms with linear or branched, saturated or unsaturated fatty acids having 2-30 carbon atoms, which can be hydroxylated; addition products of 1 to 5 propylene oxide units with mono- or polyvalent C₈₋₂₂ alkanols; addition products of at least 6 ethylene oxide and/or propylene oxide units with mono- or polyvalent C₃₋₂₂ alkanols; C₈-C₂₂ fatty alcohol esters of monovalent or polyvalent C₂-C₇ hydroxycarboxylic acids; symmetrical, asymmetrical, or cyclic esters of carbonic acid with C₃₋₂₂ alkanols, C₃₋₂₂ alkanediols, or C₃₋₂₂ alkanetriols; esters of dimers of unsaturated C₁₂-C₂₂ fatty acids (dimer fatty acids) with monovalent linear, branched, or cyclic C₂-C₁₈ alkanols or with polyvalent linear or branched C₂-C₆ alkanols; silicone oils; and mixtures of the aforementioned substances, and preferably in a total amount of from about 0.1-about 15% by weight, preferably from about 0.5-about 10% by weight, particularly preferably from about 1-about 8% by weight, extremely preferably from about 2-about 6% by weight, in each case in relation to the weight of the bleaching powder.

Further bleaching powders that are preferred as contemplated herein contain at least one polymer, which is selected from acrylic acid homo- and copolymers, methacrylic acid homo- and copolymers, itaconic acid homo- and copolymers, polysaccharides which can be chemically and/or physically modified, and mixtures of these polymers, wherein one or more of the aforementioned polymers is particularly preferably contained in a total amount of from about 0.1-about 6% by weight, preferably from about 0.5-about 4% by weight, particularly preferably from about 1-about 3.5% by weight, extremely preferably from about 2-about 3% by weight, in each case in relation to the weight of the bleaching powder.

A further subject of the present disclosure is a method for lightening keratin fibres, in particular human hair, in which a bleaching powder as contemplated herein or a bleaching powder that is preferred as contemplated herein is mixed with an oxidation composition which, in each case in relation to its weight, contains from about 50-about 96% by weight, preferably from about 70-about 93% by weight, particularly preferably from about 80-about 90% by weight of water and from about 0.5-about 20% by weight of hydrogen peroxide and also contains at least one pH adjuster in such an amount that the oxidation composition has a pH value in the range of from about 2.5 to about 5.5, measured at 20° C., is applied directly thereafter to the keratin-containing fibres, is left on the fibres for from about 5 to about 60 minutes, and then the fibres are rinsed with water and the mixture is optionally washed out using a surfactant-containing cleansing agent, wherein the bleaching powder (B) and the oxidation composition (Ox) are preferably mixed with one another in a weight-based ratio (B):(Ox) of from about 0.2-about 1, particularly preferably from about 0.3-about 0.8, more preferably from about 0.4-about 0.7, extremely preferably from about 0.5-about 0.6.

The oxidation composition (Ox) used in the lightening method as contemplated herein contains fundamentally water and hydrogen peroxide. The concentration of hydrogen peroxide is determined on the one hand by the legal requirements and on the other hand by the desired effect. It is from about 0.5-about 20% by weight, preferably from about 3-about 12% by weight, particularly preferably from about 6-about 9% by weight of hydrogen peroxide (calculated as 100% H₂O₂), in each case in relation to the weight of the oxidation composition (Ox).

The oxidation composition (Ox), in order to stabilise the hydrogen peroxide, preferably has an acidic pH value, in particular a pH value in the range of from about 2.5 to about 5.5, measured at 20° C. To stabilise the hydrogen peroxide, complexing agents, preservatives and/or buffer substances are also preferably contained.

The bleaching powder that is preferred as contemplated herein is of such a composition that the mixture with the aforementioned oxidation composition (Ox), i.e. the colour-changing agent ready for use, in particular bleaching agent, has an alkaline pH value, preferably a pH value of from about 8 to about 11.5, particularly preferably a pH value of from about 8.5 to about 11, extremely preferably a pH value of from about 9.0 to about 10.5, in each case measured at 20° C.

Oxidation compositions (Ox) used particularly preferably as contemplated herein also contain at least one oil and/or at least one fatty component having a melting point in the range of from about 23-about 110° C., preferably in a total amount of from about 0.1-about 60% by weight, particularly preferably from about 0.5-about 40% by weight, extremely preferably from about 2-about 24% by weight, in each case in relation to the weight of the oxidation composition (Ox) used with particular preference as contemplated herein. The oils suitable for the oxidation compositions (Ox) preferably used as contemplated herein are the same oils as those disclosed above as being suitable dedusting agents.

Fatty components preferably used as contemplated herein in the oxidation compositions (Ox) with a melting point in the range of from about 23-about 110° C. are selected from linear saturated 1-alkanols with 12-30 carbon atoms, preferably in a total amount of from about 0.1-about 8% by weight, particularly preferably from about 3.0 to about 6.0% by weight, in each case in relation to the weight of the oxidation composition (Ox) used as contemplated herein.

The at least one linear saturated 1-alkanol having 12-30 carbon atoms is preferably selected from lauryl alcohol, myristyl alcohol, cetyl alcohol, stearyl alcohol, arachidyl alcohol, and behenyl alcohol and also from mixtures of these 1-alkanols, particularly preferably from cetyl alcohol, stearyl alcohol and cetyl alcohol/stearyl alcohol mixtures.

Oxidation compositions (Ox) that are used with preference as contemplated herein also contain, in each case in relation to their weight, at least one linear saturated 1-alkanol having 2-30 carbon atoms in a total amount of from about 0.1-about 8% by weight, preferably in a total amount of from about 2-about 6% by weight, wherein at least one 1-alkanol selected from cetyl alcohol, stearyl alcohol and cetyl alcohol/stearyl alcohol mixtures is contained.

Further oxidation compositions (Ox) that are used with preference as contemplated herein contain at least one fatty component having a melting point in a range of from about 23-about 110° C., which is selected from esters of a saturated, monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which can be hydroxylated, candelilla wax, carnauba wax, beeswax, saturated linear C₁₄-C₃₆ carboxylic acids, and mixtures of the aforementioned substances.

Further oxidation compositions (Ox) that are preferably used as contemplated herein contain at least one surfactant or at least one emulsifier, preferably in a total amount of from about 0.5-about 10% by weight, preferably from about 1-about 5% by weight, in each case in relation to the weight of the oxidation composition (Ox) used as contemplated herein.

Surfactants and emulsifiers in the sense of the present application are amphiphilic (bifunctional) compounds that include at least one hydrophobic and at least one hydrophilic molecule part. The hydrophobic group is preferably a hydrocarbon chain having 8-28 carbon atoms, which can be saturated or unsaturated, linear or branched. This C8-C28 alkyl chain is particularly preferably linear. The basic properties of the surfactants and emulsifiers are oriented absorption at boundary surfaces and also the aggregation to micelles and the formation of lyotropic phases.

Anionic, non-ionic and cationic surfactants are particularly suitable as contemplated herein. However, zwitterionic and amphoteric surfactants are also very suitable as contemplated herein.

All anionic surface-active substances that are suitable for use on the human body are suitable as anionic surfactants in the compositions as contemplated herein. These are exemplified by a water-soluble-making anionic group, such as a carboxylate, sulfate, sulfonate or phosphate group and a lipophilic alkyl group having 8 to 30 C atoms. In addition, glycol or polyglycolether groups, ester, ether and amide groups and also hydroxyl groups can be contained in the molecule. Examples of suitable anionic surfactants are linear and branched fatty acids having 8 to 30 C atoms (soaps), alkylether carboxylic acids, acyl sarcosides, acyl taurides, acyl isethionates, sulfosuccinic acid monoesters and dialkylesters and sulfosuccinic acid mono-alkylpolyoxyethyl esters, linear alkane sulfonates, linear alpha-olefin sulfonates, alkylsulfates and alkylether sulfates and also alkyl and/or alkenyl phosphates. Preferred anionic surfactants are alkyl sulfates, alkylether sulfates and alkylether carboxylic acids each having 10 to 18 C atoms, preferably 12 to 14 C atoms in the alkyl group and up to 12 glycolether groups, preferably 2 to 6 glycol ether groups in the molecule. Examples of such surfactants are the compounds with the INCI names Sodium Laureth Sulfate, Sodium Lauryl Sulfate, Sodium Myreth Sulfate or Sodium Laureth Carboxylate.

Surface-active compounds that carry, in the molecule, at least one quaternary ammonium group and at least one carboxylate, sulfonate or sulfate group are referred to as zwitterionic surfactants. Particularly suitable zwitterionic surfactants are what are known as betaines, such as the N-alkyl-N,N-dimethylammonium glycinates, for example coco-alkyl-dimethylammonium glycinate, N-acyl-aminopropyl-N,N-dimethylammonium glycinates, for example coco-acylaminopropyl-dimethylammonium glycinate, and 2-alkyl-3-carboxymethyl-3-hydroxyethyl imidazolines, each having 8 to 18 C atoms in the alkyl or acyl group and also coco-acylaminoethylhydroxyethylcarboxymethyl glycinate. A preferred zwitterionic surfactant is the fatty acid amide derivative known under the INCI name Cocamidopropyl Betaine.

Amphoteric surfactants are understood to be surface-active compounds which, in addition to a C₈-C₂₄ alkyl or acyl group, also contain at least one free amino group and at least one —COOH or —SO₃H group in the molecule and are capable of forming inner salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkyl aminopropionic acids, and alkyl amino acetic acids each having 8 to 24 C atoms in the alkyl group. Particularly preferred amphoteric surfactants are N-coco-alkylaminopropionate, coco-acylaminoethylaminopropionate, and C₁₂-C₁₈ acyl sarcosine.

Non-ionic surfactants contain, as hydrophilic group, for example a polyol group, a polyalkylene glycol ether group or a combination of polyol group and polyglycol ether group. Such compounds are, for example, addition products of from 4 to about 50 mol ethylene oxide and/or from 0 to 5 mol propylene oxide with linear and branched fatty alcohols, with fatty acids, and with alkyl phenols, in each case having from 8 to about 20 C atoms in the alkyl group, ethoxylated mono-, di- and triglycerides, such as glycerol monolaurate+20 ethylene oxide, and glycerol monostearate+20 ethylene oxide, sorbitol fatty acid ester, and addition products of ethylene oxide with sorbitol fatty acid ester, such as Polysorbate (Tween 20, Tween 21, Tween 60, Tween 61, Tween 81), addition products of ethylene oxide with fatty acid alkanolamides and fatty amines, and alkylpolyglycosides. In particular, C₈-C₂₂ alkylmono- and oligoglycosides and ethoxylated analogues thereof and also ethylene oxide addition products with saturated or unsaturated linear fatty alcohols each having from 2 to about 30 mol of ethylene oxide per mol of fatty alcohol are suitable as non-ionic surfactants.

Further oxidation compositions used with preference as contemplated herein are exemplified in that the at least one anionic surfactant is selected from alkyl sulfates, alkyl ether sulfates, and alkyl ether carboxylic acids each having 10 to 18 C atoms, preferably 12 to 14 C atoms, in the alkyl group and up to 12 glycolether groups, preferably 2 to 6 glycol ether groups, in the molecule.

Further oxidation compositions used with preference as contemplated herein are exemplified in that at least one non-ionic surfactant, selected from ethylene oxide addition products with saturated or unsaturated linear fatty alcohols each having from 2 to about 30 mol of ethylene oxide per mol of fatty alcohol, and at least one anionic surfactant, selected from alkyl sulfates, alkylether sulfates, and alkyl ether carboxylic acids, each having 10 to 18 C atoms, preferably 12 to 14 C atoms, in the alkyl group and up to 12 glycol ether groups, preferably 2 to 6 glycol ether groups, in the molecule are contained, wherein the ratio by weight of the totality of all anionic surfactants to the totality of all non-ionic surfactants particularly preferably lies in the range of 5-50, preferably 10-30.

All cationic surface-active substances that are suitable for use on the human body are suitable in principle as cationic surfactants in oxidation compositions (Ox) used with preference as contemplated herein. These are exemplified by at least one water-soluble-making cationic group, such as a quaternary ammonium group, or by at least one water-soluble-making cationisable group, such as an amine group, and also at least one (lipophilically acting) alkyl group having 6 to 30 C atoms or at least one (lipophilically acting) imidazole group or at least one (lipophilically acting) imidazyl alkyl group.

Oxidation compositions (Ox) used with particular preference as contemplated herein contain at least one cationic surfactant, which is preferably selected from quaternary ammonium compounds having at least one C8-C24 alkyl group, esterquats and amidoamines each having at least one C8-C24 acyl group and mixtures hereof. Preferred quaternary ammonium compounds having at least one C8-C24 alkyl group are ammonium halides, in particular chlorides and ammonium alkyl sulfates, such as methosulfates or ethosulfates, such as C8-C24 alkyl trimethyl ammonium chlorides, C8-C24 dialkyl dimethyl ammonium chlorides and C8-C24 trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride, and the imidazolium compounds known under the INCI names Quaternium-27, Quaternium-83, Quaternium-87 and Quaternium-91. The alkyl chains of the above-mentioned surfactants preferably have 8 to 24 carbon atoms. Esterquats are cationic surfactants which contain both at least one ester function and at least one quaternary ammonium group as structural element and also at least one C8-C24 alkyl group or C8-C24 acyl group. Preferred esterquats are quaternised ester salts of fatty acids with triethanolamine, quaternised ester salts of fatty acids with diethanol alkylamines and quaternised ester salts of fatty acids with 1,2-dihydroxypropyldialkylamines. Such products are sold for example under the trade name Stepantex®, Dehyquart® and Armocare®. N.N-Bis(2-Palmitoyloxyethyl)dimethylammonium chloride, Distearoylethyl Dimonium Methosulfate and Distearoylethyl Hydroxyethylmonium Methosulfate are preferred examples of such esterquats.

The alkyl amidoamines are usually produced by amidation of natural or synthetic C8-C24 fatty acids and fatty acid cuts with di-(C1-C3)alkyl amino amines. A compound from this substance group which is particularly suitable as contemplated herein is stearamidopropyl dimethylamine.

Oxide compositions (Ox) used with particular preference as contemplated herein contain at least one cationic surfactant in a total amount of from about 0.01-about 5% by weight, preferably from about 0.1-about 3% by weight, particularly preferably from about 0.3-about 2% by weight, in each case in relation to the weight of the oxidation composition (Ox) used as contemplated herein.

A further subject of the present disclosure is a multi-component packaging unit (kit-of-parts) for lightening keratin fibres which contains at least two components packaged separately from one another and which is exemplified in that

i) the first component (I) is a bleaching powder as contemplated herein or is a bleaching powder that is preferred as contemplated herein,
ii) the second component (II) is an oxidation composition which contains, in each case in relation to its weight, from about 50-about 96% by weight, preferably from about 70-about 93% by weight, particularly preferably from about 80-about 90% by weight of water and from about 0.5-about 20% by weight of hydrogen peroxide and has a pH value in the range of from about 2.5 to about 5.5, measured at 20° C.,
wherein components (I) and (II) are preferably present in a weight-based ratio to one another (I):(II) of from about 0.2-about 1, particularly preferably from about 0.3-about 0.8, more preferably from about 0.4-about 0.7, extremely preferably from about 0.5-about 0.6.

A further subject of the present disclosure is a multi-component packaging unit (kit-of-parts) for changing the colour of keratin fibres, in particular human hair, containing at least three components packaged separately from one another and exemplified in that

i) the first component (I) is a bleaching powder as contemplated herein or is a bleaching powder that is preferred as contemplated herein,
ii) the second component (II) is an oxidation composition which contains, in each case in relation to its weight, from about 50-about 96% by weight, preferably from about 70-about 93% by weight, particularly preferably from about 80-about 90% by weight of water and from about 0.5-about 20% by weight of hydrogen peroxide and has a pH value in the range of from about 2.5 to about 5.5, measured at 20° C.,
iii) the third component (III) is an alkalising composition (Alk) which contains water and at least one alkalising agent, which is selected from ammonia, alkanolamines and mixtures hereof, and has a pH value in the range of from about 8-about 12, preferably of from about 9-about 11, particularly preferably of from about 9.5-about 10.5, in each case measured at 20° C., wherein the bleaching powder (B), the oxidation composition (Ox) and the alkalising composition (Alk) are preferably present in a weight-based ratio to one another (B):(Ox):(Alk) of (0.7-1.3):(2-3):(2-3), particularly preferably (0.8-1.2):(2.3-2.7):(2.3-2.7), extremely preferably 1:2:2.

A multi-component packaging unit comprises a plurality of individual components which are packaged separately from one another, and also a common packaging for these components, for example a collapsible box. The components are provided therein, each separated into different containers. Within the scope of the present disclosure, a container is understood to mean a wrapping which is present in the form of an optionally re-closable bottle, a tube, a can, a bag, a sachet or a similar wrapping. As contemplated herein, the wrapping material is not subject to any limitations. However, the wrappings are preferably made of glass or plastic.

In addition, the packaging unit can comprise application aids, such as combs, hairbrushes or paintbrushes, personal protective clothing, in particular disposable gloves, and a set of instructions.

In a further preferred embodiment as contemplated herein a bleaching powder as contemplated herein or a bleaching powder that is preferred as contemplated herein can be combined with an alkalising composition and with an oxidation composition to form a lightening, colour-changing agent for keratin fibres.

Since, when treating keratin fibres, in particular hair, with oxidising agents, in particular with hydrogen peroxide, the dye melanin, which occurs naturally in the fibres, is destroyed to a certain extent, the fibres/hair are/is inevitably lightened, i.e. the colour thereof changes even without the presence of a dye. The term “colour change” in the sense of the present application therefore includes both the lightening and colouring using one or more dyes.

The alkalising composition (Alk) used as contemplated herein contains water and at least one alkalising agent, which is selected from ammonia, alkanolamines and mixtures hereof, and which and has a pH value in the range of from about 8-about 12, preferably from about 9-about 11, particularly preferably from about 9.5-about 10.5, in each case measured at 20° C. Preferred alkanolamines are selected from monoethanolamine, 2-amino-2-methylpropanol and triethanolamine and also mixtures hereof, wherein monoethanolamine is particularly preferred. An extremely preferred alkalising agent is ammonia.

Ammonia (NH₃) in the form of its aqueous solution is usually used. Aqueous ammonia solutions contain ammonia (NH₃) often in concentrations of from about 10 to about 32% by weight. Here, the use of an aqueous ammonia solution which contains about 25% by weight ammonia (NH₃) is preferred.

Besides ammonia and alkanolamines, at least one further alkalising agent can be contained, which is selected from alkali metal silicates, alkaline earth metal silicates, alkaline earth metal hydroxide carbonates, alkaline earth metal carbonates, alkali metal metasilicates, alkaline earth metal metasilicates, alkali metal hydroxides, alkaline earth metal hydroxides and mixtures of these substances. Ammonia and/or monoethanolamine are preferably contained in the alkalising compositions used with preference as contemplated herein in amounts of from about 0.01-about 10% by weight, preferably of from about 0.1-about 7.5% by weight, more preferably of from about 0.5-about 5.5% by weight, and particularly preferably of from about 1.5-about 4.5% by weight, in each case in relation to the weight of the alkalising composition.

A further subject of the present disclosure is a method for changing the colour of keratin fibres, in particular human hair, in which a bleaching powder as contemplated herein or a bleaching powder that is preferred as contemplated herein is mixed with an oxidation composition (Ox) which contains, in each case in relation to its weight, from about 50-about 96% by weight, preferably from about 70-about 93% by weight, particularly preferably from about 80-about 90% by weight of water and from about 0.5-about 20% by weight of hydrogen peroxide and also contains at least one pH adjuster in such an amount that the oxidation composition has a pH value in the range of from about 2.5 to about 5.5, measured at 20° C., and additionally is mixed with an alkalising composition (Alk) which contains water and at least one alkalising agent which is selected from ammonia, alkanolamines and mixtures hereof, and has a pH value in the range of from about 8-about 12, preferably from about 9-about 11, particularly preferably of from about 9.5-about 10.5, in each case measured at 20° C., is applied to the keratin-containing fibres directly thereafter, is left on the fibres for from about 5 to about 60 minutes, and the fibres are then rinsed with water and the mixture is optionally washed out using a surfactant-containing cleansing agent, wherein the bleaching powder (B), the oxidation composition (Ox), and the alkalising composition (Alk) are preferably mixed with one another in a weight-based ratio (B):(Ox):(Alk) of (0.7-1.3):(2-3):(2-3), particularly preferably (0.8-1.2):(2.3-2.7):(2.3-2.7), extremely preferably 1:2:2.

As contemplated herein, the bleaching powder is preferably composed such that the mixture with the aforementioned oxidation composition (Ox) and with the aforementioned alkalising composition (Alk), i.e. the colour-changing agent ready for use, in particular the bleaching agent, has an alkaline pH value, preferably a pH value from about 8 to about 11.5, particularly preferably a pH value from about 8.5 to about 11, extremely preferably a pH value from about 9.0 to about 10.5, in each case measured at 20° C.

The ready-for-use mixtures of a bleaching powder as contemplated herein or a bleaching powder that is preferred as contemplated herein with one of the aforementioned oxidation compositions (Ox) and optionally with one of the aforementioned alkalising compositions (Alk) preferably have a viscosity in the range of from about 15,000 to about 100,000 mPas, particularly preferably from about 20,000 to about 85,000 mPas, in each case measured at 20° C. using a Brookfield viscometer, DV-II+ model, spindle 5 with a speed of 4 revolutions per minute. A viscosity in this range means that the ready-for-use agent can be easily applied and also has such a flow behaviour that this guarantees, for the agent, a sufficiently long time of action at the site of action on the keratin fibres.

In order to facilitate the miscibility of the alkalising composition used as contemplated herein with the bleaching powder as contemplated herein or the bleaching powder preferred as contemplated herein and the oxidation composition used as contemplated herein and so as to also improve the use properties of the resultant mixture that is to be used, the alkalising composition used with preference as contemplated herein preferably contains, in each case in relation to its weight, at least one surfactant in a total amount of from about 0.5-about 10% by weight, preferably from about 2-about 8% by weight.

The surfactants suitable for the alkalising compositions (Alk) used with preference as contemplated herein are selected from the same anionic, cationic, non-ionic, amphoteric and zwitterionic surfactants and emulsifiers disclosed further above as surfactants and emulsifiers suitable for the oxidation compositions (Ox) used with preference.

Alkalising compositions (Alk) that are used with particular preference as contemplated herein also contain at least one oil and/or at least one fatty component having a melting point in the range of from about 23-about 110° C., preferably in a total amount of from about 0.1-about 60% by weight, particularly preferably from about 0.5-about 40% by weight, extremely preferably from about 2-about 24% by weight, in each case in relation to the weight of the alkalising composition (Alk) used with preference as contemplated herein. The oils suitable for the alkalising compositions (Alk) used with preference as contemplated herein are the same oils disclosed further above as suitable dedusting agents.

Fatty components having a melting point in the range of from about 23-about 110° C. and used with preference in the alkalising compositions (Alk) as contemplated herein are selected from linear saturated 1-alkanols having 12-30 carbon atoms, preferably in a total amount of from about 0.1-about 20% by weight, particularly preferably from about 3-about 15% by weight, extremely preferably from about 5-about 10% by weight, in each case in relation to the weight of the alkalising composition used as contemplated herein.

The at least one linear saturated 1-alkanol having 12-30 carbon atoms is preferably selected from lauryl alcohol, myristyl alcohol, cetyl alcohol, stearyl alcohol, arachidyl alcohol, and behenyl alcohol and also from mixtures of these 1-alkanols, particularly preferably from cetyl alcohol, stearyl alcohol and cetyl alcohol/stearyl alcohol mixtures.

Alkalising compositions (Alk) used with preference as contemplated herein also contain, in each case in relation to their weight, at least one linear saturated 1-alkanol having 12-30 carbon atoms in a total amount of from about 0.1-about 20% by weight, preferably in a total amount of from about 3-about 15% by weight, extremely preferably from about 5-about 10% by weight, wherein at least one 1-alkanol, selected from cetyl alcohol, stearyl alcohol and cetyl alcohol/stearyl alcohol mixtures, is contained.

Further alkalising compositions (Alk) used with preference as contemplated herein contain at least one fatty component having a melting point in the range of from about 23-about 110° C., which is selected from esters of a saturated monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which can be hydroxylated, candelilla wax, carnauba wax, beeswax, saturated linear C₁₄-C₃₆ carboxylic acids, and mixtures of the aforementioned substances.

The bleaching powders as contemplated herein and/or the bleaching powders that are preferred as contemplated herein and/or the alkalising compositions used with preference as contemplated herein can also contain at least one substantive dye. These are dyes which are drawn directly onto the hair and do not require an oxidising process to form the colour. To dull undesirable residual colour impressions caused by melanin degradation products, in particular in the red or blue spectrum, certain substantive dyes of the complementary colours are particularly preferably contained. Substantive dyes are usually nitrophenylenediamines, nitroaminophenols, azo dyes, anthraquinones or indophenols. Substantive dyes can be anionic, cationic or non-ionic. The substantive dyes are each used preferably in an amount of from about 0.001 to about 2% by weight, in relation to the weight of the bleaching powder or the alkalising composition (Alk).

Preferred anionic substantive dyes are the compounds known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue. Preferred cationic substantive dyes include cationic triphenylmethane dyes, for example Basic Blue 7, Basic Blue 26, Basic Violet 2 and Basic Violet 14, aromatic systems which are substituted with a quaternary nitrogen group, for example Basic Yellow 57, Basic Red 76, Basic Blue 99, Basic Brown 16 and Basic Brown 17, cationic anthraquinone dyes, such as HC Blue 16 (Bluequat B), as well as substantive dyes containing a heterocyclic compound having at least one quaternary nitrogen atom, in particular Basic Yellow 87, Basic Orange 31 and Basic Red 51. The cationic substantive dyes sold under the Arianor trademark are likewise cationic substantive dyes preferred as contemplated herein. Non-ionic substantive dyes which are suitable are, in particular, non-ionic nitro and quinone dyes and neutral azo dyes. Preferred non-ionic substantive dyes include the compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-4-nitrophenol. A combination of tetrabromophenol blue and Acid Red 92 is contained very particularly preferably as contemplated herein.

As further optional ingredient, the alkalising composition used with preference as contemplated herein contains at least one oxidation dye precursor product, which is preferably selected from one or more developer components and optionally one or more coupler components.

At least one oxidation dye precursor product is particularly preferably contained in a total amount of from about 0.0001 to about 10.0% by weight, preferably from about 0.001 to about 8% by weight, in each case in relation to the weight of the alkalising composition used with preference as contemplated herein.

It may be preferred as contemplated herein to select, as developer component, at least one compound from the group formed from p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, and the physiologically acceptable salts thereof.

At least one developer component is preferably contained in a total amount of from about 0.0001 to about 10.0% by weight, preferably from about 0.001 to about 8% by weight, in each case in relation to the weight of the alkalising composition used with preference as contemplated herein.

Coupler components, within the scope of oxidative dyeing, do not alone form any significant colouration, but instead always require the presence of developer components. It is therefore preferred as contemplated herein for additionally at least one coupler component to be used when at least one developer component is used.

Coupler components that are preferred as contemplated herein are selected from 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenyl, 5-(2-hydroxyethyl)amino-2-methylphenol, 3-(diethylamino)phenol, N-cyclopentyl-3-aminophenyl, 1,3-dihydroxy-5-(methylamino)benzene, 3-ethylamino-4-methylphenol, 2,4-dichloro-3-aminophenol, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, resorcinol monomethyl ether, 2-methylresorcinol, 5-methylresorcinol, 2,5-dimethylresorcinol, 2-chlororesorcinol, 4-chlororesorcinol, pyrogallol, 1,2,4-trihydroxybenzene, 2, 6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2, 6-dimethoxypyridine, 2, 6-dihydroxy-3,4-dimethylpyridine, 3,4-diaminopyridine, 2-(2-methoxyethyl)amino-3-amino-6-methoxypyridine, 2-(4′-methoxyphenyl)amino-3-aminopyridine, 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1, 6-dihydroxynaphthalene, 1, 7-dihydroxynaphthalene, 1,8-dihydroxynaphthalene, 2,7-dihydroxynaphthalene, 2,3-dihydroxynaphthalene, 4-hydroxyindole, 6-hydroxyindole, 7-hydroxyindole, 4-hydroxyindoline, 6-hydroxyindoline, 7-hydroxyindoline, 4-6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or mixtures of these compounds or the physiologically acceptable salts thereof.

At least one coupler component is preferably contained in a total amount of from about 0.0001 to about 10.0% by weight, preferably from about 0.001 to about 8% by weight, in each case in relation to the weight of the alkalising composition used with preference as contemplated herein.

Here, developer components and coupler components are generally used in approximately equimolar amounts to one another. When the equimolar use has also proven to be expedient, a certain excess of individual oxidation dye precursors is not disadvantageous, and therefore developer components and coupler components can be contained in a molar ratio of from about 0.2-about 2, in particular from about 0.5-about 1.

The time of action is preferably from about 5 to about 60 min, in particular from about 5 to about 50 min, particularly preferably from about 10 to about 45 min. During the time in which the agents act on the fibres, it may be advantageous to assist the lightening or colour-changing process by adding heat. A phase of action at room temperature likewise corresponds to the subject matter as contemplated herein. In particular, the temperature during the time of action is between about 20° C. and about 40° C., in particular between about 25° C. and about 38° C. The agents provide good treatment results even at physiologically acceptable temperatures of less than about 45° C. After the end of the colour-changing process, all components located on the keratin fibres are rinsed from the hair using water or a surfactant-containing cleansing agent. Here, commercially available shampoo can be used in particular as cleansing agent, wherein it is then possible in particular to dispense with the cleansing agent and to carry out the rinsing process using mains water when the colour-changing agent has a higher surfactant content.

A further subject of the present disclosure is the use of a combination of cellulose 2-[2-hydroxy-3-(trimethylammonio)propoxy]ethyl ether chloride and at least one amino acid, selected from arginine, lysine, histidine or at least one of the salts of these amino acids, in a bleaching powder which contains at least one oxidising agent selected from sodium percarbonates and inorganic salts of a peroxysulfuric acid and mixtures hereof and also at least one complexing agent, selected from the following acids and/or alkali metal salts thereof: ethylenediaminetetraacetic acid (EDTA); N-hydroxyethylethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, and mixtures hereof, in a total amount of from about 0.1-about 1.4% by weight, preferably from about 0.2-about 1.4% by weight, particularly preferably from about 0.5-about 1.4% by weight, in relation to the weight of the bleaching powder, for reducing damage to keratin fibres, in particular human hair, caused by the treatment of these fibres with a mixture of the bleaching powder and an oxidation composition, which, in each case in relation to its weight, contains from about 50-about 96% by weight, preferably from about 70-about 93% by weight, particularly preferably from about 80-about 90% by weight of water and from about 0.5-about 20% by weight of hydrogen peroxide and has a pH value in the range of from about 2.5 to about 5.5, measured at 20° C.

That already said with regard to the bleaching powders as contemplated herein and the bleaching powders preferred as contemplated herein also applies, mutatis mutandis, to the multi-component packaging units (kits-of-parts) as contemplated herein and those preferred as contemplated herein.

That already said with regard to the bleaching powders as contemplated herein and the bleaching powders preferred as contemplated herein also applies, mutatis mutandis, to the methods as contemplated herein and those preferred as contemplated herein for lightening and/or changing the colour of keratin fibres.

That already said with regard to the oxidation compositions or alkalising compositions as contemplated herein and the oxidation compositions or alkalising compositions preferred as contemplated herein also applies, mutatis mutandis, to the multi-component packaging units (kits-of-parts) as contemplated herein and those preferred as contemplated herein.

That already said with regard to the oxidation compositions or alkalising compositions as contemplated herein and the oxidation compositions or alkalising compositions preferred as contemplated herein also applies, mutatis mutandis, to the methods as contemplated herein and those preferred as contemplated herein for lightening and/or changing the colour of keratin fibres.

That already said with regard to the bleaching powders as contemplated herein and the bleaching powders preferred as contemplated herein also applies, mutatis mutandis, to the use as contemplated herein.

That already said with regard to the oxidation compositions or alkalising compositions as contemplated herein and the oxidation compositions or alkalising compositions preferred as contemplated herein also applies, mutatis mutandis, to the use as contemplated herein.

### EXAMPLES

### 1.1 Developer Emulsion

| | Amount |
|---|---|
| Ingredient | (% by weight) |
| Dipicolinic acid | 0.1 |
| (2,6-dicarboxypyridine) | |
| Potassium hydroxide | 0.15 |
| Etidronic acid | 0.2 |
| Sodium cetearyl sulfate | 0.4 |
| Cetearyl alcohol | 3.5 |
| PEG-40 Castor Oil | 0.8 |
| Paraffinum liquidum | 17.0 |
| Disodium pyrophosphate | 0.1 |
| Sodium benzoate | 0.04 |
| Hydrogen peroxide | 9.0 |
| Water | to 100 |

### 1.2 Bleaching Powder Formulations

(unless specified otherwise, the values correspond to % by weight)

| | No. 1 (V) | No. 2 (E) |
|---|---|---|
| Potassium persulfate | 32.00 | 32.00 |
| Ammonium persulfate | 9.90 | 9.90 |
| L-arginine | — | 1.00 |
| Sodium silicate with SiO₂/Na₂O (molar) | 36.0 | 36.0 |
| of 2.61-2.70 | | |
| Magnesium hydroxide carbonate | 13.45 | 10.25 |
| Sodium hexametaphosphate | 0.20 | 0.20 |
| Methyl methacrylate/methacrylic acid | 1.00 | 1.00 |
| copolymer | | |
| Carboxymethyl cellulose | 2.00 | 2.00 |
| EDTA Na4 | 0.60 | 1.40 |
| Polyquaternium-10 | — | 0.47 |
| Hydrophilic silica | 0.40 | 0.40 |
| CI 77007 (Ultramarines) | 0.15 | 0.15 |
| Paraffinum Liquidum | 4.30 | 4.30 |
| Perfume | 0.60 | 0.60 |
| Total | 100.00 | 100.00 |
| Difference in tensile strength (Young's | −(1.8 ± 0.2) | −(1.3 ± 0.1) |
| modulus) after two bleaching processes | | |
| (×10⁹) | | |

The particular bleaching powder and developer emulsion were mixed with one another in a ratio by weight of 1:2.

All bleaching treatments lead to a loss of tensile strength of the hair fibres. This loss of tensile strength, however, can be statistically significantly reduced with the composition E2 as contemplated herein.

### 2. Application

100 g of the freshly produced mixture of the particular bleaching powder and the developer emulsion were applied to dry strands of hair (4 g of mixture per gram of hair). Once the strands had been bleached for 45 minutes at 32° C., they were washed for 2 minutes using water and dried using a hairdryer.

This bleaching process was repeated once so that the strands were bleached a total of two times in succession.

### 3. Measurements of the Hair Tensile Strength

### Background

Young's modulus is also known as the modulus of elasticity (E-modulus). It corresponds to the ratio of stress to strain with linear elastic behaviour (in Hooke's region).

Hooke's law of elasticity says that the longitudinal change of a body (strain) is linearly dependent on the force causing the deformation (stress).

For moist hair, the linear correlation for strain is from about 0 to about 2%.

Young's modulus is a measure for the strength of a fibre (the higher the value of Young's modulus, the stronger is the fibre).

The strands used for the measurements included 40 fibres (Kerling International (Backnang, Germany), European Natural Hair 7/0; batch #2014, blend 138).

The mean cross-sectional area of each individual hair was first determined (universal dimensions-measuring device UDM 5000A (Zimmer GmbH, Darmstadt)), more specifically at a temperature of 22° C. and a relative air humidity of 50%. These values are required for the calculation of the stress values.

### 3.2. Determination of Young's Modulus Prior to the Application of the Bleaching Agent

All hair fibres were soaked for one hour in water. They were then stretched (0-1.5% extension) with the stress-strain system MTT 680 with control unit UV 1000 (Dia-Stron Ltd, UK) at a constant speed of 10 mm/min within the elastic phase. The modulus of elasticity (Young's modulus) was then calculated (Software: UvWin 1.32.1000 (Dia-Stron Ltd, UK).

### 3.3. Determination of Young's Modulus Following the Application of the Bleaching Agent

After the four bleaching treatments, the hair fibres were stored for at least 48 hours. The hair fibres were soaked for one hour in water. They were then stretched (0-1.5% extension) with the stress-strain system MTT 680 with control unit UV 1000 (Dia-Stron Ltd, UK) at a constant speed of 10 mm/min within the elastic phase. The modulus of elasticity (Young's modulus) was then calculated (Software: UvWin 1.32.1000 (Dia-Stron Ltd, UK).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A bleaching powder, comprising
a) at least one oxidising agent selected from sodium percarbonates, inorganic salts of a peroxysulfuric acid, or mixtures hereof,
b) cellulose 2-[2-hydroxy-3-(trimethylammonio)propoxy]ethyl ether chloride,
c) at least one amino acid; selected from arginine, lysine, histidine, or at least one of the salts of these amino acids, and
d) at least one complexing agent selected from the following acids and/or alkali metal salts thereof: ethylenediaminetetraacetic acid (EDTA); N-hydroxyethylethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder.

2. The bleaching powder according to claim 1, wherein the at least one oxidising agent a) is contained in a total amount of 5-85% by weight, in relation to the total weight of the bleaching powder.

3. The bleaching powder according to claim 1, wherein the cellulose 2-[2-hydroxy-3-(trimethylammonio)propoxy]ethyl ether chloride is contained in a total amount of from about 0.01-about 1.00% by weight in relation to the total weight of the bleaching powder.

4. The bleaching powder according to claim 1, further comprising at least one dicarboxylic acid having 2 to 10 carbon atoms.

5. The bleaching powder according to claim 1, wherein the at least one amino acid selected from arginine, lysine, histidine, or at least one salt of these amino acids, is contained in a total amount, converted to the mass of free amino acid, of from about 0.1-about 7% by weight in relation to the total weight of the bleaching powder.

6. The bleaching powder according to claim 1, comprising, in each case in relation to the total weight of the bleaching powder, from about 0.01-about 1.00% by weight of cellulose 2-[2-hydroxy-3-(trimethylammonio)propoxy]ethyl ether chloride, and a total amount, converted to the mass of the free amino acid, of from about 0.1-about 7% of the at least one amino acid selected from arginine, lysine, histidine, or at least one salt of these amino acids.

7. The bleaching powder according to claim 1, comprising water in an amount of from 0 to about 8% by weight in relation to the total weight of the bleaching powder.

8. The bleaching powder according to claim 1, wherein the at least one oxidising agent a) is selected from sodium peroxydisulfate, potassium peroxydisulfate, ammonium peroxydisulfate, sodium percarbonate, sodium peroxymonosulfate, potassium peroxymonosulfate, ammonium peroxymonosulfate, and mixtures of these compounds.

9. The bleaching powder according to claim 1, further comprising at least one oil present in a total amount of from about 0.1-about 15% by weight in relation to the total weight of the bleaching powder.

10. A method for lightening keratin fibres, wherein the method comprises:
mixing a bleaching powder with an oxidation composition, wherein the bleaching powder comprises:
a) at least one oxidising agent selected from sodium percarbonates, inorganic salts of a peroxysulfuric acid, or mixtures hereof,
b) cellulose 2-[2-hydroxy-3-(trimethylammonio)propoxy]ethyl ether chloride,
c) at least one amino acid selected from arginine, lysine, histidine, or at least one of the salts of these amino acids, and
d) at least one complexing agent selected from the following acids and/or alkali metal salts thereof: ethylenediaminetetraacetic acid (EDTA); N-hydroxyethylethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder, and
wherein the oxidation composition comprises, in each case in relation to its weight, from about 50-about 96% by weight of hydrogen peroxide, at least one pH adjustor in such an amount that the oxidation composition has a pH value in the range of from about 2.5 to about 5.5, measured at 20° C.,
applying the mixture of the bleaching powder and the oxidation composition to the keratin comprising fibres directly after mixing, wherein the mixture is left on the fibres for from about 5 to about 60 minutes, and
rinsing the fibres with water with the mixture optionally washed out using a surfactant-containing cleansing agent.

11. A multi-component packaging unit (kit-of-parts) for lightening keratin fibres, comprising at least two components packaged separately from one another, wherein i) the first component (I) is a bleaching powder comprising
a) at least one oxidising agent selected from sodium percarbonates, inorganic salts of a peroxysulfuric acid, or mixtures hereof,
b) cellulose 2-[2-hydroxy-3-(trimethylammonio)propoxy]ethyl ether chloride,
c) at least one amino acid selected from arginine, lysine, histidine, or at least one of the salts of these amino acids, and
d) at least one complexing agent selected from the following acids and/or alkali metal salts thereof: ethylenediaminetetraacetic acid (EDTA); N-hydroxyethylethylenediaminetriacetic acid; aminotrimethylenephosphonic acid; diethylenetriaminepentaacetic acid; lauroyl ethylenediaminetriacetic acid; nitrilotriacetic acid; iminodisuccinic acid; N-2-hydroxyethyliminodiacetic acid; ethylene glycol-bis-(beta-aminoethyl ether)-N,N-tetraacetic acid; amino trimethylene phosphonic acid, pentasodium amino trimethylene phosphonate, or mixtures hereof, in a total amount of from about 0.1-about 1.4% by weight in relation to the total weight of the bleaching powder,
ii) the second component (II) is an oxidation composition which comprises, in each case in relation to its weight, from about 50-about 96% by weight of water and from about 0.5-about 20% by weight of hydrogen peroxide and has a pH value in the range of from about 2.5 to about 5.5, measured at 20° C.

12. (canceled)

13. (canceled)

14. (canceled)

15. A bleaching powder according to claim 1, wherein the at least one oxidising agent a) is contained in a total amount of from about 20-about 55% by weight in relation to the total weight of the bleaching powder, and wherein the d) at least one complexing agent is present in a total amount of from about 0.5-1.4% by weight in relation to the total weight of the bleaching powder.

16. The bleaching powder according to claim 1, wherein the cellulose 2-[2-hydroxy-3-(trimethylammonio)propoxy]ethyl ether chloride is present in an amount of from about 0.30-about 0.47% by weight in relation to the total weight of the bleaching powder, and wherein the at least one amino acid selected from arginine, lysine, histidine, or at least one salt of these amino acids is present in a total amount, converted to the mass of free amino acid, of from about 1-about 2% by weight in relation to the total weight of the bleaching powder.

17. The bleaching powder according to claim 4, wherein the at least one dicarboxylic acid having 2 to 10 carbon atoms is selected from succinic acid, malic acid, oxalic acid, malonic acid, adipic acid, pimelic acid, suberic acid, azelaic acid, sebacic acid, maleic acid, fumaric acid, D-tartaric acid, L-tartaric acid, meso-tartaric acid, racemic acid, alpha-ketoglutaric acid, beta-ketoglutaric acid, oxaloacetic acid and/or at least one salt of these acids.

18. The bleaching powder according to claim 4, wherein the at least one dicarboxylic acid having 2 to 10 carbon atoms is present in a total amount, converted to the mass of free dicarboxylic acid, of from about 0.03-about 7% by weight in relation to the total weight of the bleaching powder.

19. The bleaching powder according to claim 4, wherein the at least one dicarboxylic acid having 2 to 10 carbon atoms is present in a total amount, converted to the mass of free dicarboxylic acid, of from about 0.9-about 1.5% by weight in relation to the total weight of the bleaching powder.

20. The bleaching powder according to claim 1, wherein the at least one amino acid selected from arginine, lysine, histidine, or at least one salt of these amino acids, is present in a total amount, converted to the mass of free amino acid, of from about 1-about 2% by weight in relation to the total weight of the bleaching powder.

21. The bleaching powder according to claim 1, comprising water in an amount of from about 0.5 to about 3% by weight in relation to the total weight of the bleaching powder.

22. The bleaching powder according to claim 9, wherein the at least one oil is present in a total amount of from about 2-about 6% by weight in relation to the total weight of the bleaching powder.

23. The multi-component packaging unit of claim 11, further comprising a third component (III) packages separately from the first component (I) and the second component (II), wherein the third component (III) is an alkalising composition (Alk) which comprises water and at least one alkalising agent selected from ammonia, alkanolamines and mixtures hereof, and has a pH value in the range of from about 8-about 12, measured at 20° C.,
